(19) United States
(12) Patent Application Publication
(10) Pub. No.: US 2022/0180408 A1
(43) Pub. Date: Jun. 9, 2022

(54) QUANTIFICATION OF HUMAN FOOD TASTES ACROSS FOOD MATRICES, FOOD SERVERS AND FOOD CONSUMERS

(71) Applicants: Zhiguo Zhou, Chapel Hill, NC (US); Yu Ye, Morrisville, NC (US)

(72) Inventors: Zhiguo Zhou, Chapel Hill, NC (US); Yu Ye, Morrisville, NC (US)

(21) Appl. No.: 17/113,079

(22) Filed: Dec. 6, 2020

(51) Int. Cl. G06Q 30/02; G16H 40/67; G16H 20/60; G06Q 30/06; G06Q 50/12; G06Q 10/06; G06F 16/28

(52) U.S. Cl. CPC G06Q 30/0282; G06Q 30/0201; G16H 40/67; G16H 20/60; G06F 16/285; G06Q 50/12; G06Q 30/0218; G06Q 10/06315; G06Q 30/0631

(57) ABSTRACT

A method, system, and computer program product are described for quantitation of human food tastes. Such quantitation involves taste calibration of human food tasters, to constitute an initiation cohort of trained tasters who then taste and quantitatively score food items, from which a computing device determination of a normative distributized score for the food items is transmitted to user devices for quantitative guidance in selection of food and food providers, with food consumers receiving such quantitative guidance subsequently electively providing consumer scoring to enhance consistency and reliability of the food taste quantitation.

### FIELD

The present disclosure relates to systems, methods, and computer program products for quantification of human food tastes.

### DESCRIPTION OF THE RELATED ART

In the context of increasing worldwide culinary sophistication, globalization of food and ingredient markets, diversification of diets, and proliferation of dining options, there is a persistent and continuing need to characterize food for human consumption.

The impetus for food characterization is related to personal preferences for specific tastes in food that is consumed, to enable consumers to select foods for consumption that are congruent with such personal preferences, to achieve sensory satisfaction, and dietary, nutrition, and health goals. Correlatively, food preparers and providers seek to accommodate such preferences in the food products that they commercialize.

In the restaurant business, menus may vary over time and locations to greater or lesser degrees depending on the type and character of the restaurant, food sources, ingredient availability, costs, eating trends, etc., but there is generally a fundamental focus on achieving consistency in taste of menu items on such menus. The efforts to achieve consistency are based on the goals of restaurant owners and employees to be cost-effective and efficient in the repeated performance of recipes and associated sourcing of food ingredients, as well as the desire to attract patrons on the basis of experiences communicated by prior customers regarding the food offerings of the restaurant, and the desire to gain repeat business, in which customer expectations of food taste are satisfied.

Such efforts to achieve consistency in taste of menu items are also shaped by the social and environmental necessity of reducing food waste and carbon footprint by precisely matching food tastes to customer preferences. Currently, an estimated 1.3 billion tons of food, corresponding to approximately 30% of global production, with associated economic, environmental, and social costs of $2.6 trillion, is lost or wasted annually, according to the United Nations Food and Agricultural Organization (http://www.fao.org/policy-support/policy-themes/food-loss-food-waste/en/, accessed Sep. 13, 2020). A significant percentage, estimated to be at least 10%, of such food waste is attributable to a mismatching of food tastes and individual food consumer preferences. A correction of such mismatching correspondingly has the potential to substantially reduce food loss and wastage, thereby preserving the resources that otherwise are used to produce such lost and wasted food, as well as achieving environmental benefits, since food production is attributed to be responsible for 26% of global greenhouse gas emissions (https://ourworldindata.org/food-ghg-emissions, accessed Sep. 13, 2020), and since usage of fertilizers and pesticides used in producing food that is subsequently lost or wasted increases the environmental burden, e.g., pollution, of our planet.

In addition to the above, changes in sensory perception of food taste are increasingly recognized as a correlate of a number of disease states and physiological conditions, including hepatitis B, diabetes, and SARS-CoV-2. This raises the possibility of utilizing such changes in sensory perception of food taste as a pre-diagnosis tool for such disease states and physiological conditions.

A need therefore exists for systems and methods for quantitating human food tastes to address and resolve the foregoing problems.

### SUMMARY

The present disclosure relates to systems, methods, and computer program products for quantitating human food tastes.

In one aspect, the disclosure relates to a computer-implemented method of quantitating human food tastes, comprising: (a) receiving, by a computing device comprising a processor and memory, taste scores from an initiation cohort of human food tasters tasting one or more taste calibration food items at a taste calibration station; (b) determining, by the computing device, a normative distributized calibration score for each of the one or more taste calibration food items at the taste calibration station that have been taste-scored by the initiation cohort of human food tasters, to specify a qualified initiation cohort of human food tasters; (c) receiving, by the computing device, taste scores from the qualified initiation cohort of human food tasters tasting one or more offered food items of one or more food sources; (d) determining, by the computing device, a normative distributized score for the one or more offered food items of the one or food sources by the qualified initiation cohort of human food tasters; (e) providing, by the computing device, to user interfaces of user devices, the normative distributized menu item score for the one or more offered food items of the one or more food sources; (f) receiving, by the computing device, offered food item scores sent by users of the user devices for the one or more offered food items of the one or more food sources that have been tasted by the users; (g) determining, by the computing device, from the offered food item scores sent by users from the user devices for the one or more offered food items, an updated normative distributized offered food item score, when the menu item scores sent by the users from the user devices satisfy one or more predetermined qualification conditions for inclusion; and (h) providing, by the computing device, to the user interfaces of the user devices, the updated normative distributized offered food item score for the one or more offered food items of the one or more food sources.

In another aspect, the disclosure relates to a system for quantitating human food tastes, comprising: a CPU, a computer readable memory and a computer readable storage medium associated with a computing device; program instructions to obtain taste scores from an initiation cohort of human food tasters tasting one or more taste calibration food items at a taste calibration station; program instructions to determine a normative distributized calibration score for each of the one or more taste calibration food items at the taste calibration station that have been taste-scored by the initiation cohort of human food tasters, to specify a qualified initiation cohort of human food tasters; program instructions to obtain taste scores from the qualified initiation cohort of human food tasters tasting one or more offered food items of one or more food sources; program instructions to determine a normative distributized score for the one or more offered food items of the one or food sources by the qualified initiation cohort of human food tasters; program instructions to provide to user interfaces of user devices connected to the computing device via a network, the normative distributized menu item score for the one or more offered food items of the one or more food sources; program instructions for obtain from the user devices offered food item scores for the one or more offered food items of the one or more food sources that have been tasted by the users; program instructions to determine from the offered food item scores sent by users from the user devices for the one or more offered food items, an updated normative distributized offered food item score, when the menu item scores sent by the users from the user devices satisfy one or more predetermined qualification conditions for inclusion; and program instructions to provide to the user interfaces of the user devices, the updated normative distributized offered food item score for the one or more offered food items of the one or more food sources.

In a further aspect, the disclosure relates to a computer program product for quantitating human food tastes, the computer program product comprising a computer readable storage medium having program instructions embodied therewith, the program instructions executable by a computing device to cause the computing device to: obtain taste scores from an initiation cohort of human food tasters tasting one or more taste calibration food items at a taste calibration station; determine a normative distributized calibration score for each of the one or more taste calibration food items at the taste calibration station that have been taste-scored by the initiation cohort of human food tasters, to specify a qualified initiation cohort of human food tasters; obtain taste scores from the qualified initiation cohort of human food tasters tasting one or more offered food items of one or more food sources; determine a normative distributized score for the one or more offered food items of the one or food sources by the qualified initiation cohort of human food tasters; provide to user interfaces of user devices connected to the computing device via a network, and the normative distributized menu item score for the one or more offered food items of the one or more food sources; obtain from the user devices offered food item scores for the one or more offered food items of the one or more food sources that have been tasted by the users; determine from the offered food item scores sent by users from the user devices for the one or more offered food items, an updated normative distributized offered food item score, when the menu item scores sent by the users from the user devices satisfy one or more predetermined qualification conditions for inclusion; and provide to the user interfaces of the user devices, and the updated normative distributized offered food item score for the one or more offered food items of the one or more food sources.

Other aspects, features and embodiments of the disclosure will be more fully apparent from the ensuing description and appended claims.

### BRIEF DESCRIPTION OF THE DRAWINGS

FIG. 1 is a chart of illustrative human food tastes and exemplified taste markers.

FIG. 2 is a schematic flow chart, showing component operations of a system and method of the present disclosure, in one illustrative embodiment thereof.

FIG. 3 is a schematic representation of a networked communications system, wherein a networked communications server is operatively linked in communication relationship with a device of an initiation cohort of human food tasters and with devices of patrons to whom normative distributized determinations of restaurant menu item taste scores are communicated and from whom patron taste scoring data are communicated to the server as input for refinement of normative distributized determinations of restaurant menu item taste scores distributed to recipient devices of the system.

### DETAILED DESCRIPTION

The present disclosure relates to human food taste quantitation systems, methods, and computer program products.

As used herein, the singular forms “a”, “and”, and “the” include plural referents unless the context clearly dictates otherwise.

As used herein, a normative distributized score is a score that is automatically determined in real time by a computing device based on inputted score data that is processed by the computing device to generate a normative distribution of score data, excluding score data that are incompatible with the normative distribution. The normative distribution may for example be a Gaussian distribution, a step function distribution, or other suitable data distribution, with respect to which the incompatible score data are excluded.

In one aspect, the disclosure relates to a computer-implemented method of quantitating human food tastes, comprising: (a) receiving, by a computing device comprising a processor and memory, taste scores from an initiation cohort of human food tasters tasting one or more taste calibration food items at a taste calibration station; (b) determining, by the computing device, a normative distributized calibration score for each of the one or more taste calibration food items at the taste calibration station that have been taste-scored by the initiation cohort of human food tasters, to specify a qualified initiation cohort of human food tasters; (c) receiving, by the computing device, taste scores from the qualified initiation cohort of human food tasters tasting one or more offered food items of one or more food sources; (d) determining, by the computing device, a normative distributized score for the one or more offered food items of the one or food sources by the qualified initiation cohort of human food tasters; (e) providing, by the computing device, to user interfaces of user devices, the normative distributized menu item score for the one or more offered food items of the one or more food sources; (f) receiving, by the computing device, offered food item scores sent by users of the user devices for the one or more offered food items of the one or more food sources that have been tasted by the users; (g) determining, by the computing device, from the offered food item scores sent by users from the user devices for the one or more offered food items, an updated normative distributized offered food item score, when the menu item scores sent by the users from the user devices satisfy one or more predetermined qualification conditions for inclusion; and (h) providing, by the computing device, to the user interfaces of the user devices, the updated normative distributized offered food item score for the one or more offered food items of the one or more food sources.

In various embodiments of the method of the present disclosure, the one or more food sources include restaurants and the one or more offered food items are menu items of such restaurants. Alternatively, the food sources may be food stores, food trucks, farm stands, farmers' markets, food delivery services, catering businesses, personal chef businesses, street food vendors, cocktails and other drinks and bars, homemade food using recipes and cookbooks, or other sources of foods and food products.

The normative distributized offered food item scores in various embodiments of the method of the present disclosure may be single value numerical scores, or alternatively may be numerical value ranges. The taste scores and normative distributized offered food item scores may in various embodiments be scored in predetermined taste categories, such as one or more, or all, of taste categories such as for example spiciness, saltiness, sweetness, sourness, and bitterness.

Step (b) of the method of the present disclosure, of determining, by the computing device, a normative distributized calibration score for each of the one or more taste calibration food items at the taste calibration station that have been taste-scored by the initiation cohort of human food tasters, to specify a qualified initiation cohort of human food tasters, may further include, in various embodiments, determining deviations from the normative distributized calibration score disqualifying individuals from further participation in the initiation cohort, so that individuals whose taste scoring selections are vastly different from those of other individuals in the initiation cohort, representing taste scores that are inconsistent with the normative distributized calibration scores of the initiation cohort are deselected in specifying the qualified initiation cohort of human food tasters. In such manner, the qualified initiation cohort represents a group of individuals who are fully vetted and trained at the taste calibration station to provide meaningful score data as members of the qualified initiation cohort.

In step (g) of the method of the present disclosure, namely, determining, by the computing device, from the offered food item scores sent by users from the user devices for the one or more offered food items, an updated normative distributized offered food item score, when the menu item scores sent by the users from the user devices satisfy one or more predetermined qualification conditions for inclusion, the one or more predetermined qualification conditions may include a maximum allowable quantitative deviation of each offered food item score sent by the users, in relation to an existing normative distributized score for such offered food item, so that grossly inconsistent outlier scores are excluded from scoring consideration.

In the method of the present disclosure, the user device may be of any suitable type, and may for example be a tablet device, laptop device, desktop device, smart phone device, or other device of appropriate character.

The method of the present disclosure may be carried out in various embodiments in which when offered food item scores sent by users are below predetermined minimum values or are above predetermined maximum values, in relation to an existing normative distributized score for such offered food items, the computing device automatically transmits to the user interfaces of the user devices a notification of potential adverse physiological conditions associated with such offered food item scores sent by the users. Thus, for example, a change of taste, or scoring indicative of an absence of taste may have health implications, since many diseases, e.g., hepatitis B, diabetes, and SARS-CoV-2, have initial symptoms including taste changes, and notifications of potential adverse physiological conditions associated with the food item scores sent by the users may serve an important interest in combating such diseases.

The computing device in the method of the present disclosure may be constituted, programmed, and arranged to provide various additional information to the users connected via a network with the computing device.

For example, in the performance of the method, the computing device may provide at least one of food source recommendations and diet recommendations to the interfaces of the user devices based on offered food item scores previously sent by the users of the user devices.

As another example, the computing device may be constituted, programmed, and arranged to provide group food source recommendations to multiple user devices of respective users self-identifying to the computing device as constituting a group, wherein the group food source recommendations are based on offered food item scores previously sent by the users in the group to the computing device.

As a still further example, the computing device may be constituted, programmed, and arranged to correlate a candidate offered food item based on ingredients thereof, with normative distributized offered food item scores previously determined for offered food items containing such ingredients, and computationally determine a predictive normative distributized score for such candidate offered food item that is automatically transmitted by the computing device to the user interfaces of the user devices.

The computing device in the method of the present disclosure, in various embodiments thereof, may include software provided as a service in a cloud environment, to the user devices.

The method of the present disclosure may be carried out, in a further embodiment, to comprise receiving, by the computing device, from the one or food sources an identification of ingredients of the one or more offered food items of the one or more food sources, such computing device responsively computationally determining a correspondence of ingredient amounts to normative distributized scores for the one or more offered food items containing the ingredients, and providing the correspondence to the user interfaces of the user devices. The computer device may for example provide the correspondence to the user interfaces of the user devices together with at least one of appertaining food source recommendations and appertaining diet recommendations. As another example, the computing device may provide the correspondence to the user interfaces of the user devices together with appertaining health-related information.

In a further implementation of the method of the present disclosure, the computing device may be constituted, programmed, and arranged to identify new food sources to the user interfaces of the user devices, as offering food items corresponding to the one or more offered food items of the one or more food sources for which normative distributized offered food item scores have been provided by the computing device to the user interfaces of the user devices.

Further implementations of the method of the present disclosure, as variously described herein, may additionally comprise any of: (i) implementations of the method, wherein the computing device is configured to identify demographic information of the users based on their offered food item scores and to transmit such demographic information to a food producer or food preparer for guidance in design and production of new food items; (ii) implementations of the method, as further comprising receiving, by a vendor food source comprised in the one or more food sources, on a vendor device, user food item scores and/or taste profiles based thereon, sent by the user devices of users ordering from or dining at the vendor food source, with the vendor food source responsively offering one or food items to users of the user devices based on the user food item scores and/or taste profiles, individually or groupwise, and when groupwise, with or without preference to some users in the group; (iii) implementations of the method, further comprising receiving, by a vendor food source comprised in the one or more food sources, on a vendor device, normative distributized offered food item scores and/or taste profiles based thereon, for a predetermined population, sent by the computing device, with the vendor food source responsively generating a new food offering or recipe based on the distributized offered food item scores and/or taste profiles based thereon, for the predetermined population; (iv) implementations of the method, further comprising receiving, by a fruit or vegetable producer food source comprised in the one or more food sources, on a producer device, normative distributized offered food item scores and/or taste profiles based thereon, for a predetermined population, sent by the computing device, with the fruit or vegetable producer food source responsively timing pickup or delivery of fruits or vegetables, for the predetermined population; and (v) implementations of the method, further comprising receiving on device(s), by the one or more food sources, normative distributized offered food item scores and/or taste profiles based thereon, for a predetermined population, for guidance of the one or more food sources in meeting customer taste preferences. In the foregoing implementations (i)-(v), the specified device or devices of vendors, producers, and food sources may be of any suitable type, and may for example be a tablet device, laptop device, desktop device, smart phone device, or other device of appropriate character.

In another aspect, the disclosure relates to a system for quantitating human food tastes, comprising: a CPU, a computer readable memory and a computer readable storage medium associated with a computing device; program instructions to obtain taste scores from an initiation cohort of human food tasters tasting one or more taste calibration food items at a taste calibration station; program instructions to determine a normative distributized calibration score for each of the one or more taste calibration food items at the taste calibration station that have been taste-scored by the initiation cohort of human food tasters, to specify a qualified initiation cohort of human food tasters; program instructions to obtain taste scores from the qualified initiation cohort of human food tasters tasting one or more offered food items of one or more food sources; program instructions to determine a normative distributized score for the one or more offered food items of the one or food sources by the qualified initiation cohort of human food tasters; program instructions to provide to user interfaces of user devices connected to the computing device via a network, the normative distributized menu item score for the one or more offered food items of the one or more food sources; program instructions for obtain from the user devices offered food item scores for the one or more offered food items of the one or more food sources that have been tasted by the users; program instructions to determine from the offered food item scores sent by users from the user devices for the one or more offered food items, an updated normative distributized offered food item score, when the menu item scores sent by the users from the user devices satisfy one or more predetermined qualification conditions for inclusion; and program instructions to provide to the user interfaces of the user devices, the updated normative distributized offered food item score for the one or more offered food items of the one or more food sources.

In a further aspect, the disclosure relates to a computer program product for quantitating human food tastes, the computer program product comprising a computer readable storage medium having program instructions embodied therewith, the program instructions executable by a computing device to cause the computing device to: obtain taste scores from an initiation cohort of human food tasters tasting one or more taste calibration food items at a taste calibration station; determine a normative distributized calibration score for each of the one or more taste calibration food items at the taste calibration station that have been taste-scored by the initiation cohort of human food tasters, to specify a qualified initiation cohort of human food tasters; obtain taste scores from the qualified initiation cohort of human food tasters tasting one or more offered food items of one or more food sources; determine a normative distributized score for the one or more offered food items of the one or food sources by the qualified initiation cohort of human food tasters; provide to user interfaces of user devices connected to the computing device via a network, the normative distributized menu item score for the one or more offered food items of the one or more food sources; obtain from the user devices offered food item scores for the one or more offered food items of the one or more food sources that have been tasted by the users; determine from the offered food item scores sent by users from the user devices for the one or more offered food items, an updated normative distributized offered food item score, when the menu item scores sent by the users from the user devices satisfy one or more predetermined qualification conditions for inclusion; and provide to the user interfaces of the user devices, the updated normative distributized offered food item score for the one or more offered food items of the one or more food sources.

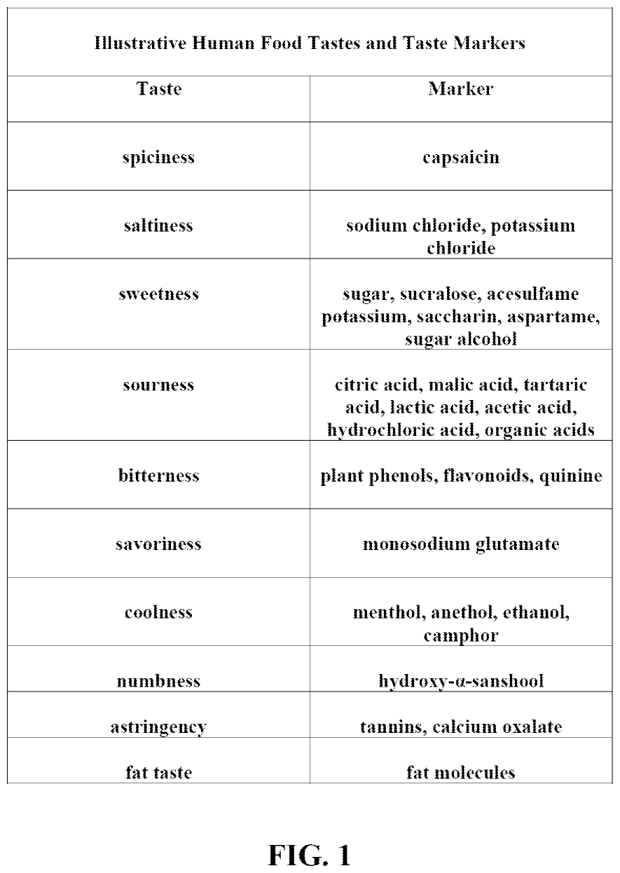

FIG. 1 is a chart of illustrative human food tastes and taste markers. The method, system, and computer program product of the present disclosure may be implemented to provide quantitation of any tastes of the spectrum of human food tastes, and the specific tastes identified in FIG. 1, of spiciness, saltiness, sweetness, sourness, and bitterness are merely illustrative of tastes that may be quantitated in the broad practice of the present disclosure. The human food tastes may be quantitated with respect to particular markers associated with such tastes, a marker in such context referring to a chemical compound, ingredient, or material that exhibits a selected taste characteristic. The marker may be employed as a taste determinant if actually present in the specific food item being scored, or the marker may be employed as a referential standard, such as capsaicin, which has various associated heat or spiciness scales. The taste scoring of food items in the quantitation performed in the practice of the present disclosure thus may be normalized or referentialized to the taste marker, at concentrations or amounts of interest.

In addition to spiciness, and spiciness marker capsaicin, FIG. 1 identifies the tastes of: saltiness, with associated markers sodium chloride and potassium chloride; sweetness, with associated markers sugar, sucralose, acesulfame potassium, saccharin, aspartame, and sugar alcohol; sourness, with associated markers citric acid, malic acid, tartaric acid, lactic acid, acetic acid, hydrochloric acid, and organic acids; bitterness, with associated markers plant phenols, flavonoids, and quinine; savoriness, with associated marker monosodium glutamate; coolness, with associated markers menthol, anethol, ethanol, and camphor; numbness, with associated marker hydroxy-α-sanshool; astringency, with associated markers tannins and calcium oxalate; and fat taste, with associated marker fat molecules.

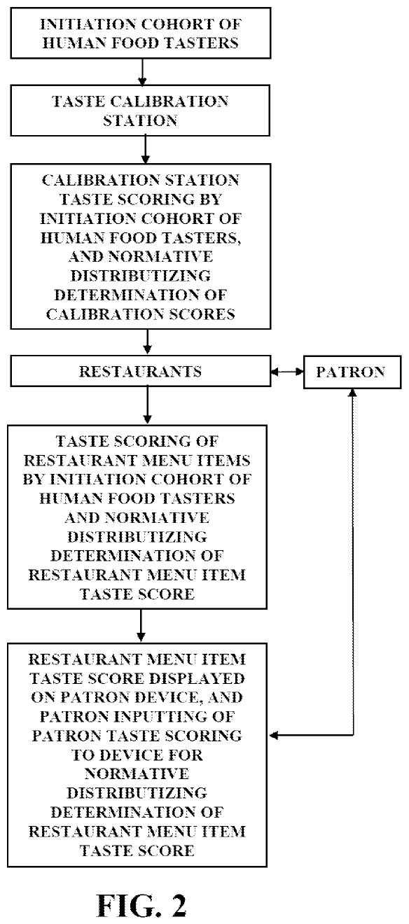

FIG. 2 is a schematic flow chart, showing component operations of a system and method of the present disclosure, in one illustrative embodiment thereof.

As shown in FIG. 2, an initiation cohort of human food tasters may be assembled, as individuals that are selected from among candidate human food tasters based on relevant selection criteria, e.g., diet history, self-identified taste spectrum and acuity, and preliminary taste tests.

The initiation cohort of human food tasters, once assembled, is specifically trained to quantitative taste scoring at a taste calibration station. The taste calibration station may be a specifically established tasting facility, or a restaurant having a menu of appropriately diverse taste characteristics, or other food-service or dining facility selected for use as the taste calibration station.

At the taste calibration station, members of the initiation cohort of human food tasters are served specific food offerings that are taste-scored by cohort members. These food offering taste scores then are provided to a computing device, which may be a server established for performing the method of the present disclosure, including a CPU, a computer readable memory and a computer readable storage medium associated with a computing device. The taste scores specified by the initiation cohort of human food tasters may be transmitted by cohort members directly to the computing device of the server by cohort member devices, e.g., mobile devices, connected to the computing device via a network. Alternatively, the taste scores may be compiled at the taste calibration station, and transmitted from a taste calibration station device to the computing device of the networked server. Regardless of the manner and mode of transmission, the computing device obtains the calibration station taste scoring of the initiation cohort members, and the computing device responsively determines a normative distributizing calibration score for each of the food items tasted by cohort members at the taste calibration station.

The cohort members, or selected one or ones thereof, may be subjected to additional taste scoring exercises at the taste calibration station, as required to fully acclimate the cohort members to the taste scoring quantitation methodology, with subsequent determinations by the computing device of normative distributizing calibration scores being generated and confirmed to produce a normative distribution for the initiation cohort as a whole, such as a Gaussian distribution of taste scores producing a consensus average value or range of values that implicate a satisfactory sensory familiarity with food items to be subsequently scored, and consistency of the scoring effort, by the members of the initiation cohort.

By such operational training, the initiation cohort of human food tasters then is prepared to visit food providers, e.g., restaurants, in the present example. Initiation cohort members then visit the restaurants offering the restaurant menu items to be taste-scored, and based on tasting of such restaurant menu items assess same with a restaurant menu item taste score for each of the menu items. These taste scores then are obtained by the computing device, e.g., from mobile devices employed by the initiation cohort members for such purpose, to which the initiation cohort members input their respective taste scores for the restaurant menu items, for transmission by the mobile devices to the computing device of the server. The computing device responsively determines a normative distributizing calibration score for each of the food items tasted by cohort members at the restaurants, based on initiation cohort members scoring inputs. The normative distributizing calibration scores for the scored food items at the specified restaurants are then provided by the computing device, as restaurant menu item taste scores, to user interfaces of user devices, e.g., mobile devices such as smart phones or smart watches, or laptop or desktop computers, of potential patrons of the specified restaurants. The user devices for such purpose are suitably networked in communication with the computing device of the server, e.g., pursuant to a software as a service (SaaS) subscription, or other appropriate arrangement. The potential patrons thus may be notified of restaurant menu item taste scores, enabling such potential patrons to select any of the specified restaurants for patronage, based on the restaurant menu item taste scores for such restaurants.

The restaurant menu item taste scores may be numeric or numerical range scores in any of the previously identified taste categories. For example, a specific menu food item may have a spiciness score of 7 and a saltiness score of 5 on respective 20 point scales, or another specific menu food item may have a sweetness score of 10-12 on a 15 point scale, so that potential patrons are provided with useful taste score information for menu items of potential interest.

Based on such restaurant menu items taste scores, a patron may visit the restaurant to sample the menu items selected by the patron. The patron then may input to the patron's user device, e.g., smart phone, smartwatch, laptop, or desktop, a patron taste score for the tasted menu item in the selected taste categories. The computing device of the server communicationally linked to the patron's user device thereupon obtains from the user device the menu item scores determined by the patron for the menu items that have been tasted by the patron, and the computing device responsively determines, using the patron food scores, an updated normative distributized menu item score reflecting such patron food scores, when the transmitted patron food scores satisfy one or more predetermined qualification conditions for inclusion. If satisfying such qualification conditions, updated normative distributized restaurant menu item taste scores are computationally determined by the computing device of the server, and the updated normative distributized restaurant menu item taste scores are provided by the computing device to user interfaces of user devices that are communicationally linked to the computing device via a networked communications system.

The predetermined qualification conditions are programmatically applied by the computing device of the server to rule-in or rule-out the patron menu items taste scores, and may be of any suitable type or collection. For example, the predetermined qualification conditions may include a maximum allowable quantitative deviation of each patron menu item taste score, in relation to an existing normative distributized score for such offered food item, so that greatly outlying patron taste scores are automatically excluded by the computing device from updating consideration.

By such automatic exclusion according to predetermined qualification conditions, grossly inaccurate taste scores from a novice patron acclimating to the quantitation scheme of the present disclosure are eliminated, and as such patron becomes increasingly familiar with such quantitation scheme and scores menu items in a manner that satisfies the inclusion criteria for the normative distributized scoring quantitation scheme, the patron can make ongoing and substantial contribution to the quantitative reliability and consistency of the food taste quantification methodology of the present disclosure.

Accordingly, it is seen that the food taste quantification methodology of the present disclosure utilizes a front-end trained initiation cohort of human food tasters to establish a taste scoring database from which normative distributized taste scores are determined and disseminated to user devices of prospective patrons, and such scoring database is progressively augmented by participation of prospective patrons to become actual patrons and input corresponding scoring data to the database from which updated normative distributized taste scores are determined and disseminated to user devices.

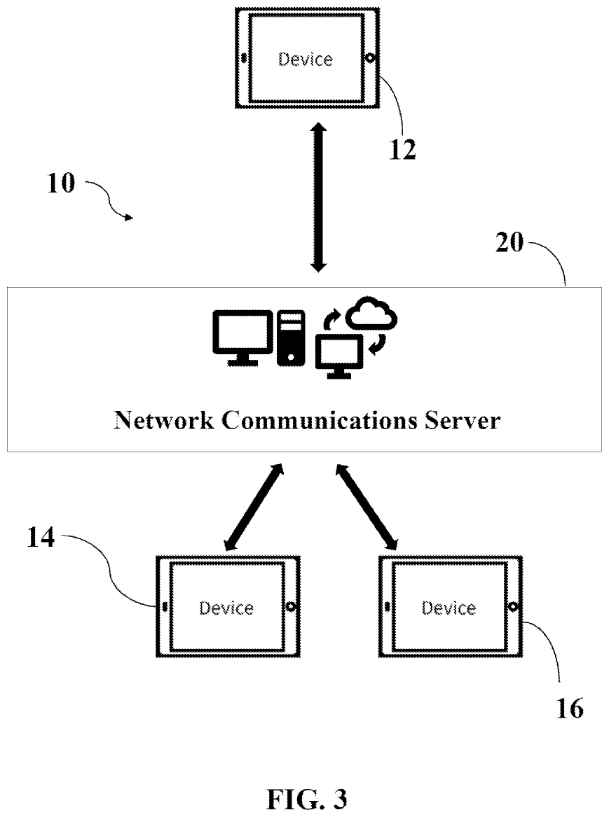

FIG. 3 is a schematic representation of a networked communications system 10, wherein a networked communication server 20 is operatively linked in communication relationship with (i) a user device 12 of an individual member of an initiation cohort of human food tasters and (ii), concurrently or subsequently, with devices 14, 16 of patrons to whom normative distributized determinations of restaurant menu item taste scores are communicated and from whom patron taste scoring data are communicated to the server 20 as input for refinement of normative distributized determinations of restaurant menu item taste scores distributed to recipient devices of the system including patron devices 14, 16.

The following is a further example of the operation of the methodology of the present disclosure.

In such further example, human food tasters are calibrated and trained using food with reliably consistent tastes in a first step. Selected human food tasters are sent to eat at a designated place, the taste calibration station, where a taste is consistently served in a broad range so that taste scores can be established by the human food tasters. For example, a human food taster may be asked to eat at an establishment serving Buffalo wings with a taste of spiciness determined to encompass a 17-point scale, i.e., a spiciness score of 1-17, with 17 being the spiciest. Selection of the taste calibration station may be flexible as long as it has a demonstrated consistency in taste of menu items, a broad range of taste of menu items, and ready accessibility by tasters, and is preferably popular across a large geographic area, although such popularity and geographic extent are not absolute requirements, so long as the taste calibration station meets the other aforementioned criteria. Alternatively, the calibration at the taste calibration station can be conducted using homemade or catered food with the same qualifications identified above (consistency in taste of menu items, a broad range of taste of menu items, and ready accessibility by tasters).

At the end of the calibration, the human food tasters need to graduate by scoring all blinded tastes at the taste calibration center correctly with only minor deviations. This calibration test will be repeated during the second step, described below, as often as is needed to ensure the human food taster memory of the taste scale at the taste calibration station is fresh and consistent in all human food tasters. Additionally, the number of human food tasters should be large enough to cover each taste scale.

In a second step, food tastes are scored using trained human food tasters against the scale at the taste calibration station. The human food tasters trained in the first step will be sent to other restaurants to taste dishes on the menu and report taste scores by comparing their tastes to those at the taste calibration station. The scores are reported via an application (iTaste) that is developed for smart phone input and display of food taste scores. The application provides an interface to (1) collect and upload data to a server, where taste scores for each dish are calculated, and (2) display the calculated food taste scores to general customers.

The population of tasters needs to be sufficiently large (e.g., >11 for each dish) so that taste scores on each dish form a normal distribution. Outliers that fall too far out of the normal distribution are eliminated and remaining scores are averaged to set the taste scores of each dish.

To further control quality of data, some calibrating food with known or established taste scale numbers used in the first step may be sent to human food tasters in the second step, without disclosure of taste scale numbers to the human food tasters. The human food tasters would be asked to score such calibrating food at the same time as they score food from other restaurants. Comparison between the human food tasters' reported taste scores and known taste scores would provide a referential basis for determining the accuracy of human food tasters' scores on food from other restaurants.

Additionally, non-personally identifiable information of each human food taster (e.g., home ZIP Code, age, race, gender, etc.) and restaurant information (e.g., name, location, and dish names) would also be collected in the application.

At the end of the second step, a large number of dishes at selected restaurants are scored with a spiciness number. Each of the human food tasters is assigned a spiciness taste number.

In a third step, tuning and expansion of food taste scores is carried out using motivated common food consumers.

Following the trained human food tasters, common food consumers, identified as any food eater (but not a trained human food taster in the prior steps of the method), is granted free access to the iTaste application identifying the taste scores determined in the second step. The common food consumers can voluntarily provide feedback on food taste scores at any restaurant they try. There feedback would be focused on the range of tastes in which they are specifically interested. Initially, incentives such as food credits may be provided, to motivate common food consumers who taste food that is already scored by the human food tasters. To control the quality of feedback from common food consumers, their initial responses are not counted. Only after common food consumers provide multiple scores that are consistent with those of the human food tasters for the same dishes, are the scores of the common food consumers counted for determination of application scores, as those common food consumers become familiar with the taste scoring system. The subsequent scorings of the common food consumers could be statistically weighted to tune the scores that already exist or expand scoring to new dishes in a new geographic area.

This three-step food scoring method can establish taste scores of any food in any area of the world. The taste calibration station thus is introduced as a standardizing ruler for digitalizing food tastes.

Trained human food tasters calibrated by the taste calibration station experience may be employed to compare taste calibration stations in differing geographic areas as an aid to coordinate food scoring in such differing geographic areas. For example, taste scores in the United States could be established using one set of taste calibration stations, while in China, another set of taste calibration stations could be utilized. The respective scoring systems could be linked together, using the same human food tasters at the respective taste calibration stations.

The three-step food scoring method could be employed across different food matrices (the term matrices referring to recipes, compositions, formulations, and ingredients of food), with human food tasters and common food consumers being employed as analytical components for quantifying tastes of various food matrices.

This three-step food scoring method may be employed to motivate food servers, human food tasters, and common food consumers for extensive participation in the quantification of human food tastes. The scoring method uses consumers to directly score food, and may be implemented to include food comparisons using chemical analysis methods. Chemical analysis methods may be employed, independent of consideration of other taste-influencing factors such as temperature and texture of food as well as the presence of other ingredients in the food.

The scoring method may be employed to establish correlations between food taste and food ingredients.

In an illustrative implementation, recipes of scored food would be collected in the iTaste application. Access to recipes may be secured by open source availability or negotiation with restaurants or other food preparers. Based on the collected recipes, taste ingredient concentrations can be calculated using weights of ingredients that render the taste, based on total weight of food, e.g., 0.5 g dried red guajillo chilies in 500 g of chili soup. Alternatively, concentrations of taste ingredients, such as capsaicin, sodium/potassium chloride, and sugar, can be measured experimentally and used to calculate taste concentrations.

In these recipes, food matrices (e.g., soup, chicken wings, etc.) and food preparation processes, particularly including steps that may degrade the taste ingredients (for example, heating at 375° F. for 10 minutes) would also be recorded in detail.

Initially, food ingredients and food preparation processes would be linked to score food tastes on a one-to-one basis. As the database is expanded, tastes may be predicted with new recipes. As a simplest example, if a new recipe were exactly the same as an existing recipe in the application database, its taste scores would be exactly the same as those of the old recipe. The score would be corrected in the same manner as existing dishes that are similarly prepared. In many instances, the correlation between taste score and recipes is continuous, such that if a new recipe were written with ingredients or processing procedures being intermediate between two prior recipes, the corresponding taste score would also be intermediate between the taste scores of the two prior recipes.

The relationship between the recipe and the taste score can be rigorously determined using machine learning as the database is expanded, to generate a model that accurately predicts taste scores from recipes.

In like manner, correlations may be established between food taste and taste molecule concentrations. Food taste molecules can be measured using various published analytical methods or by use of analytical methods that may be developed for such purpose. Food taste molecules can be linked to food taste scores on a one-to-one basis. As the database is expanded, taste may be predicted with the concentration of taste molecules. In a simplest example, if a new concentration were exactly the same as an existing concentration in the database, its taste score would be exactly the same as that of the prior concentration. In many instances, the correlation between taste scores and taste molecule concentrations is continuous, so that if a new concentration of a taste molecule intermediate between concentrations of the same taste molecule in two prior recipes is employed, the taste score will likewise be intermediate between the taste scores of the two prior recipes. The relationship between taste molecule concentration and taste score may be rigorously determined using machine learning as the database is expanded. In many cases, taste scales are exponentially correlated to the concentration of taste molecules. After the modeling is established, taste molecule concentrations can be calculated from taste scores and utilized to identify health concerns and provide corresponding notifications.

Thus, food taste scores may be employed to identify, notify, and address health concerns. After a correlation between a food recipe and taste scores established, concentration of taste molecules can be calculated based on taste scores. For example, salt concentration can be calculated from saltiness scores. Additionally, salt consumption can be monitored over a period of time by the iTaste system and method of the present disclosure, to provide notifications and warnings of long-term risks. Subsequently, a cumulative total amount of salt that consumers had consumed could be immediately identified and compared to the US FDA recommendations regarding salt intake. The iTaste application can then warn food consumers when the amount of taste molecules (e.g., sugar, or salt) exceeded the recommended intakes for dietary health, with suggestion for intake of foods having taste scores that balance both health and dining enjoyment. Instead of checking food ingredients and calculating, the use of taste scores can be utilized to connect consumer health directly and quantitatively to consumer tastes, to inform the consumer of approaches to healthy eating that are consistent with their individual tastes.

The methodology of the present disclosure can be utilized to calculate an optimal group taste score. In order to accommodate taste preferences of a group of individuals who share food and intend to compromise individual preferences in favor of group satisfaction, and optimal group taste score can be determined by adding each individual taste score as weighted by an appropriate weighting factor in the iTaste application. A group coordinator, who is aware of which ones of the group need to be satisfied the most or least, may allocate appropriate weighting factors, e.g., generating a group taste score as a sum of the respective individual taste scores as multiplied by the appropriate weighting factor for that individual.

Taste-taste and taste-matrix interactions may be utilized in the implementation of the methodology of the present disclosure. Using the food taste scoring system, a determination of taste-taste and taste-matrix interactions is facilitated. For example, the mechanism of influences on taste scores of food matrices with respect to molecular or ingredient components that affect but do not render taste directly can be elucidated by utilization of the food taste scoring system. By way of illustration, the concepts of “heartiness”, “full flavor”, and “richness” are embodied in the Japanese term “kokumi”, in reference to compounds in food that lack their own taste, but nonetheless intensify or enhance basic taste characteristics of food such as sweetness, sourness, saltiness, bitterness, or savory character, e.g., garlic when employed as a flavor ingredient for intensification or enhancement of such basic taste characteristics.

Based on qualified taste results, an optimal order of food serving for the most favorable consumer experience can be identified, e.g., identification of a food to accommodate a taste of an individual exceeding the tolerance of general consumers. For example, a determination that saltiness at a score of 10 and above would dramatically increase spiciness but that the spiciness would not affect saltiness at all, may be employed to specify that the spiciness dish be served before the dish with saltiness at 10 and above, since the spiciness and saltiness of such subsequent dish would not be affected.

By quantification of food tastes in accordance with the present methodology, consumers are able to be more certain about the tastes of food they order or prepare, particularly when they travel and/or attempt a new recipe. Such quantification may avoid unpleasant surprises by the tastes of similar dishes at different places due to different recipes or ingredients. Accordingly, consumers would avoid expenditures of time and money for food that they may otherwise not like.

In addition, quantification of food tastes in accordance with the present methodology affords substantial benefit to persons who suffer dietary diseases such as diabetes or high blood pressure, who can be correspondingly informed before consuming meals that may worsen health concerns. Consumers thereby would have knowledge of quantified tastes on which food choices favorable to their health and appetites can be based, thereby improving satisfaction by balancing of taste and health considerations.

As a further benefit, quantification of food tastes in accordance with the present methodology may be employed for guidance to food industries to meet consumer taste preferences. From knowledge of taste scores desired by customers at the time of ordering or reservation, food vendors and suppliers are enabled to personalize food tastes to meet taste demands of individual customers. When food needs to be supplied to a group, a vendor or supplier can choose food with tastes that best accommodate the group, either with or without preference to specific individuals in the group. Such additional service focus would distinguish food vendors and suppliers who use the quantitative food scoring methodology, who would thereby gain more customers. Further, peer pressure and free market forces may also function to motivate food vendors and suppliers to implement the quantitative food scoring methodology, to gain a competitive advantage in their businesses.

The benefits of quantification of food tastes also extend to global food chains and restaurants in the introduction of new dishes to specific geographic areas, in which a taste profile of the population of the area from an iTaste database would provide a fundamentally important referential basis for creation of new recipes for such dishes.

Quantified food tastes additionally have benefit to produce suppliers and distributors, who can be guided by quantitated taste levels, e.g., of sweetness of specific fruits, that are preferred in the localities that they service, so that harvesting and delivery of produce occurs at a timing that accommodates such preferred quantitated taste levels.

As mentioned hereinabove, many diseases, such as hepatitis B, diabetes, and SARS-CoV-2 exhibit initial symptoms involving taste changes, and attention to differences in subjective taste quantitation and consensus quantitation taste scores can be utilized to tentatively diagnose diseases, and application system notifications can be correspondingly generated and transmitted to consumers who thereby are alerted to seek prompt medical attention.

Still further, substantial reductions in waste of food and in carbon footprint as well as in pesticide and fertilizer usage may be achieved by precision matching of food tastes to customer preferences, using quantitated taste scoring. In specific embodiments, reductions of food waste at restaurants after implementation of quantitative taste scores can be used as one of key business indicators reflecting the merit of the methodology of the present disclosure. Such methodology enables food savings, and savings of resources allocated to production of food, as well as reduction of the greenhouse gas emissions that are associated with food production. Such methodology additionally entails benefits in reducing healthcare expenditures and increasing health and longevity by precision matching of food taste to consumer preferences.

It will therefore be appreciated that the method, system, and computer program product of the present disclosure function to provide quantitative information to accurately describe tastes of food to food consumers before purchasing or consuming such food. Such quantitative information enables food consumers to dramatically improve their satisfaction rates with their food purchase and consumption.

The disclosure, as variously set out herein in respect of features, aspects and embodiments thereof, may in particular implementations be constituted as comprising, consisting, or consisting essentially of, some or all of such features, aspects and embodiments, as well as elements and components thereof being aggregated to constitute various further implementations of the disclosure. The disclosure is set out herein in various embodiments, and with reference to various features and aspects of the disclosure. The disclosure contemplates such features, aspects and embodiments in various permutations and combinations, as being within the scope of the invention. The disclosure may therefore be specified as comprising, consisting or consisting essentially of, any of such combinations and permutations of these specific features, aspects and embodiments, or a selected one or ones thereof.

Further, while the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

What is claimed is:

1. A computer-implemented method of quantitating human food tastes, comprising:
(a) receiving, by a computing device comprising a processor and memory, taste scores from an initiation cohort of human food tasters tasting one or more taste calibration food items at a taste calibration station;
(b) determining, by the computing device, a normative distributized calibration score for each of said one or more taste calibration food items at said taste calibration station that have been taste-scored by said initiation cohort of human food tasters, to specify a qualified initiation cohort of human food tasters;
(c) receiving, by the computing device, taste scores from said qualified initiation cohort of human food tasters tasting one or more offered food items of one or more food sources;
(d) determining, by the computing device, a normative distributized score for said one or more offered food items of said one or food sources by said qualified initiation cohort of human food tasters;
(e) providing, by the computing device, to user interfaces of user devices, the normative distributized menu item score for said one or more offered food items of said one or more food sources;
(f) receiving, by the computing device, offered food item scores sent by users of said user devices for said one or more offered food items of said one or more food sources that have been tasted by said users;
(g) determining, by the computing device, from said offered food item scores sent by users from said user devices for said one or more offered food items, an updated normative distributized offered food item score, when said menu item scores sent by said users from said user devices satisfy one or more predetermined qualification conditions for inclusion; and
(h) providing, by the computing device, to said user interfaces of said user devices, the updated normative distributized offered food item score for said one or more offered food items of said one or more food sources.

2. The method of claim 1, wherein said one or more food sources include restaurants and said one or more offered food items are menu items of said restaurants.

3. The method of claim 1, wherein the normative distributized offered food item scores are single value numerical scores.

4. The method of claim 1, wherein the normative distributized offered food item scores are numerical value ranges.

5. The method of claim 1, wherein said taste scores and normative distributized offered food item scores are scored in predetermined taste categories.

6. The method of claim 5, wherein said predetermined taste categories include at least one of spiciness, saltiness, sweetness, sourness, and bitterness.

7. The method of claim 5, wherein said predetermined taste categories include spiciness, saltiness, sweetness, sourness, and bitterness.

8. The method of claim 1, wherein said one or more predetermined qualification conditions in (g) includes a maximum allowable quantitative deviation of each offered food item score sent by said users, in relation to an existing normative distributized score for such offered food item.

9. The method of claim 8, wherein the user device is a tablet or smart phone device.

10. The method of claim 1, wherein when offered food item scores sent by users are below predetermined minimum values or are above predetermined maximum values, in relation to an existing normative distributized score for such offered food items, the computing device automatically transmits to the user interfaces of the user devices a notification of potential adverse physiological conditions associated with such offered food item scores sent by said users.

11. The method of claim 1, wherein the computing device provides at least one of food source recommendations and diet recommendations to said interfaces of said user devices based on offered food item scores previously sent by the users of said user devices.

12. The method of claim 1, wherein the computing device provides group food source recommendations to multiple user devices of respective users self-identifying to the computing device as constituting a group, wherein said group food source recommendations are based on offered food item scores previously sent by the users in said group to the computing device.

13. The method of claim 1, wherein the computing device correlates a candidate offered food item based on ingredients thereof, with normative distributized offered food item scores previously determined for offered food items containing said ingredients, and computationally determines a predictive normative distributized score for said candidate offered food item that is automatically transmitted by the computing device to the user interfaces of the user devices.

14. The method of claim 1, wherein the computing device includes software provided as a service in a cloud environment, to said user devices.

15. The method of claim 1, further comprising receiving, by the computing device, from said one or food sources an identification of ingredients of said one or more offered food items of said one or more food sources, said computing device responsively computationally determining a correspondence of ingredient amounts to normative distributized scores for said one or more offered food items containing said ingredients, and providing said correspondence to said user interfaces of said user devices.

16. The method of claim 15, wherein the computing device provides said correspondence to said user interfaces of said user devices together with at least one of appertaining food source recommendations and appertaining diet recommendations.

17. The method of claim 15, wherein the computing device provides said correspondence to said user interfaces of said user devices together with appertaining health-related information.

18. The method of claim 1, wherein the computing device identifies new food sources to said user interfaces of said user devices, as offering food items corresponding to said one or more offered food items of said one or more food sources for which normative distributized offered food item scores have been provided by the computing device to said user interfaces of said user devices.

19. The method of claim 1, wherein the computing device is configured to identify demographic information of said users based on their offered food item scores and to transmit such demographic information to a food producer or food preparer for guidance in design and production of new food items.

20. The method of claim 1, further comprising receiving, by a vendor food source comprised in said one or more food sources, on a vendor device, user food item scores and/or taste profiles based thereon, sent by said user devices of users ordering from or dining at the vendor food source, with the vendor food source responsively offering one or food items to users of said user devices based on said user food item scores and/or taste profiles, individually or groupwise, and when groupwise, with or without preference to some users in the group.

21. The method of claim 1, further comprising receiving, by a vendor food source comprised in said one or more food sources, on a vendor device, normative distributized offered food item scores and/or taste profiles based thereon, for a predetermined population, sent by the computing device, with the vendor food source responsively generating a new food offering or recipe based on said distributized offered food item scores and/or taste profiles based thereon, for said predetermined population.

22. The method of claim 1, further comprising receiving, by a fruit or vegetable producer food source comprised in said one or more food sources, on a producer device, normative distributized offered food item scores and/or taste profiles based thereon, for a predetermined population, sent by the computing device, with the fruit or vegetable producer food source responsively timing pickup or delivery of fruits or vegetables, for said predetermined population.

23. The method of claim 1, further comprising receiving on device(s), by said one or more food sources, normative distributized offered food item scores and/or taste profiles based thereon, for a predetermined population, for guidance of said one or more food sources in meeting customer taste preferences.

24. A system for quantitating human food tastes, comprising:
a CPU, a computer readable memory and a computer readable storage medium associated with a computing device;
program instructions to obtain taste scores from an initiation cohort of human food tasters tasting one or more taste calibration food items at a taste calibration station;
program instructions to determine a normative distributized calibration score for each of said one or more taste calibration food items at said taste calibration station that have been taste-scored by said initiation cohort of human food tasters, to specify a qualified initiation cohort of human food tasters;
program instructions to obtain taste scores from said qualified initiation cohort of human food tasters tasting one or more offered food items of one or more food sources;
program instructions to determine a normative distributized score for said one or more offered food items of said one or food sources by said qualified initiation cohort of human food tasters;
program instructions to provide to user interfaces of user devices connected to said computing device via a network, the normative distributized menu item score for said one or more offered food items of said one or more food sources;
program instructions for obtain from said user devices offered food item scores for said one or more offered food items of said one or more food sources that have been tasted by said users;
program instructions to determine from said offered food item scores sent by users from said user devices for said one or more offered food items, an updated normative distributized offered food item score, when said menu item scores sent by said users from said user devices satisfy one or more predetermined qualification conditions for inclusion; and
program instructions to provide to said user interfaces of said user devices, the updated normative distributized offered food item score for said one or more offered food items of said one or more food sources.

25. A computer program product for quantitating human food tastes, the computer program product comprising a computer readable storage medium having program instructions embodied therewith, the program instructions executable by a computing device to cause the computing device to:
obtain taste scores from an initiation cohort of human food tasters tasting one or more taste calibration food items at a taste calibration station;
determine a normative distributized calibration score for each of said one or more taste calibration food items at said taste calibration station that have been taste-scored by said initiation cohort of human food tasters, to specify a qualified initiation cohort of human food tasters;
obtain taste scores from said qualified initiation cohort of human food tasters tasting one or more offered food items of one or more food sources;
determine a normative distributized score for said one or more offered food items of said one or food sources by said qualified initiation cohort of human food tasters;
provide to user interfaces of user devices connected to said computing device via a network, the normative distributized menu item score for said one or more offered food items of said one or more food sources;
obtain from said user devices offered food item scores for said one or more offered food items of said one or more food sources that have been tasted by said users;
determine from said offered food item scores sent by users from said user devices for said one or more offered food items, an updated normative distributized offered food item score, when said menu item scores sent by said users from said user devices satisfy one or more predetermined qualification conditions for inclusion; and
provide to said user interfaces of said user devices, the updated normative distributized offered food item score for said one or more offered food items of said one or more food sources.
